(19) United States
(12) Patent Application Publication
(10) Pub. No.: US 2007/0281891 A1
(43) Pub. Date: Dec. 6, 2007

(54) Lock solution for medical devices

(76) Inventor: Anders Wieslander, LUND (SE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/629,784

(22) PCT Filed: Jun. 15, 2005
(86) PCT No.: PCT/SE05/00912

§ 371 Date: May 15, 2007

Related U.S. Application Data
(60) Provisional application No. 60/580,502, filed on Jun. 17, 2004.

(30) Foreign Application Priority Data
Jun. 17, 2004 (SE) 0401568-1

(51) Int. Cl. A61K 31/70; A61K 31/366; A61K 31/35; A61K 31/11; A61K 31/34

(52) U.S. Cl. 514/23; 514/460; 514/473; 514/693

(57) ABSTRACT

The invention relates to a lock solution for medical devices comprising carbohydrates and/or glucose degradation products as antimicrobial agent(s).

### TECHNICAL FIELD OF THE INVENTION

The present invention relates to a lock solution for medical devices.

Furthermore, the present invention relates to a device for applying the lock solution of the invention in a catheter or other system for access to an organism, a vascular system, tissue structures or hollow organs. The invention also relates to a method for applying the lock solution in a catheter or other system for access.

### BACKGROUND ART

Intravascular catheter related bloodstream infections are an important cause of illness and excess medical cost. From a clinical point of view health care professionals, i. e. physicians and nurses have limited possibilities to decrease the risks for infections at sides of vascular access even when they take great care in aseptic procedures. A small amount of bacteria, which would not be a problem in a bloodstream, could grow in the catheter. Resistance of bacteria to antibiotics is also attributed to the biofilm formation of adhered bacteria which could not be penetrated in full depth by antibiotics.

Catheters and especially chronic venous catheters have a number of drawbacks. The significant drawbacks are that such catheters can become occluded by thrombus and biofilm formation. In order to prevent clotting of and biofilm formation in catheters in blood vessels between uses, e.g. between dialysis treatments when the catheter is not perfused by blood and dwells inside a vein, the lumens of the catheter are often filled with a lock solution. As used herein, the term “lock solution” refers to a solution that is injected or infused into a lumen of a catheter between treatments or into other system for access to an organism, a vascular system, tissue structures or hollow organs, in order prevent formation of clots and biofilm layers on the surface thereof.

Based on these findings there is a clear medical need to design a lock solution, especially in catheters or port systems, preventing bacterial growth and by this biofilm formation and preventing bioincompatible reactions, especially formation of clots and fibrin or platelets deposits. The importance of antimicrobial activity and preventing of clot formation in the catheter has been addressed in a paper by Wang et. al. (J. of infectious diseases, 1993, 167:39-36), in a paper of Rodney M. Donlan; Emerging Infectious Diseases, 2001, Vol. 7, No. 2, and a paper of Klaus Konner, J Nephrol, 2002, 15 (supl. 6), S28-S32 where a strong relation is described between platelets deposition and promotion of bacterial growth.

Basically there are different methods to prevent the risk of bacteria growth and biofilm formation and to prevent clotting in catheters: antimicrobial modification of catheter surfaces, e.g. according to PCT/SE2004/000804, which hereby is incorporated by reference; anticoagulatoric modification of catheter surfaces; application of lock solution with heparin; application of lock solutions with antimicrobial substances, e.g. antibiotics, taurolidine and citrate.

To reduce the incidence of infections in medical devices a lock solution is commonly used by first flushing the catheter with saline to remove, e. g. blood from the catheter lumen. Subsequently, an anticoagulant solution, typically heparin, is injected to displace the saline and to fill the lumen. A lock solution of heparin excludes blood from the lumen at the same time as it actively inhibits clotting and formation of thrombus within the lumen. Combinations of the lock solution with various antimicrobial substances have also been suggested in order to inhibit infections and thrombus formation at the same time.

However, heparin has a number of drawbacks. The procedure to prepare a heparin solution after each catheter session is time consuming and presents a risk for bleeding, contamination and dosing errors by physicians or nurses. Patients in intravenous therapy or treated by hemodialysis and hemofiltration have to be subjected to such treatments several times a week. The need to combine a separate antimicrobial agent in the lock solution complicates the procedure further and is costly.

EP 1040841 A1 relates to a lock solution containing taurolidine, taurultam or a mixture thereof, which solution is used for preventing thrombosis formation and/or bacterial growth on a liquid-containing surface of a liquid delivery system.

US 20010003746 A1 discloses use of a composition comprising at least one taurinamide derivative, and at least one compound selected from the group consisting of biologically acceptable acids and biologically acceptable salts thereof, for inhibiting or preventing infection and blood coagulation in or near a medical prosthetic device after the device has been inserted in a patient.

US20030144362 A1 relates to an antibacterial formulation comprising an antibacterial agent having relatively low viscosity, e.g. alcohols, iodine and tauroline, which agent is mixed with viscosity increasing agent.

WO02/05873 A2 discloses devices, methods and kits for use in connection with catheters. More particularly, devices, methods and kits for infusion of a liquid into a catheter are described, e.g. a transcutaneous catheter, wherein a lock solution is infused into a catheter for preventing occlusion and for inhibiting infections. Alternatively a saline solution is infused into an indwelling catheter to flush the contents of the catheter from the distal end of the catheter.

U.S. Pat. No. 6,423,050 relates to a central-vein catheter locked by anticoagulant and bactericidal solutions separated by an air bubble which prevents mixing of the solutions. A multi chamber syringe facilitates sequential injection of the anticoagulant, air and bactericidal agent with only one connection in order to decrease the risks for contamination.

WO02/05188 A1 relates to an implanted catheter locked with a solution comprising a lower alcohol and an additive comprising an antimicrobial, e.g. taurolidine or triclosan, or an anticoagulant, typically riboflavine, sodium citrate, ethylene diamin tetraacetic acid, or citric acid. Furthermore, the use of taurolidine may result in increased frequency of clots depositions in the catheter in comparison with heparin lock solutions. The application of antibiotics is also very costly.

U.S. Pat. No. 5,433,705 discloses an antiinfection catheter arrangement with a rigid or flexible tube with a connection piece at the distal end. The catheter has a filling and a suction device which can be attached to the connection piece and one or more active principle reservoirs with a total volume equal to the capacity of the catheter. This volume is entirely filled with a substance containing at least an antibiotic agent or a chemotherapeutic agent or an antiviral agent, preferably aminoglycoside.

For the reasons stated above it would be highly desirable to provide an improved lock solution and a device and method for locking implanted catheters between subsequent applications. Desirably such a lock solution should prevent bacterial growth and by this biofilm formation and also prevent bioincompatible reactions, especially formation of clots and fibrin or platelets deposits. Furthermore, the locking method and device should prevent contamination of the catheter lumen in order to reduce the risk for infections.

### SUMMARY OF THE INVENTION

It is therefore an object of the present invention to provide a lock solution, wherein the drawbacks mentioned above are eliminated or at least alleviated.

This object has been achieved by a lock solution for medical devices characterised in that the lock solution comprises carbohydrates and/or glucose degradation products.

Preferred embodiments of the lock solution are provided in claims 2-9.

According to one embodiment of the invention the carbohydrates are chosen form the group of glucose and/or fructose.

According to a preferred embodiment of the invention the glucose degradation products are chosen from the group of 3-deoxyglucosone (3-DG), acetaldehyde, formaldehyde, acetaldehyde, glyoxal, methylglyoxal, 5-hydroxymethyl-2-furaldehyde (5-HMF), 2-furaldehyde, and 3,4-dideoxyglucosone-3-ene (3,4-DGE).

In another embodiment of the invention the lock solution comprises the carbohydrates and/or the glucose degradation products as the sole antimicrobial agent(s) in the lock solution.

In another embodiment of the invention the lock solution further comprises an anticoagulation agent, preferably citrate.

In yet another embodiment the concentration of citrate in the lock solution is <4 weight % and in yet another embodiment the concentration of carbohydrates are 0.1-50 weight %.

In another embodiment the concentration of the glucose degradation products in the lock solution are the following: 3-90 μM 3,4-3,4-dideoxyglucosone-3-ene, 15-1800 μM 3-deoxyglucosone and 2-900 μM 5-hydroxymethyl-2-furaldehyde.

The lock solution of the present invention contains only substances with known methabolic pathways. It may be used in small volumes but at high concentrations. In this way major systemic toxicity or bioincompatibility reactions are excluded.

The mixture of carbohydrates with other additives combine anticoagulatory and antimicrobial properties without exerting toxicity for the organism.

A specific object of the invention is to provide a pre-filled applicator device for applying the lock solution of the invention in a catheter or other access system, which ensures enhanced operative simplicity during aseptic handling and which reduces the risk of microbial, particle or air contamination.

Another object of the invention is to provide a pre-filled applicator which makes possible a simple procedure for rinsing and locking a catheter or other access system, which ensures enhanced operative simplicity during aseptic handling and which reduces the risk of microbial, particle or air contamination.

According to the invention, these and other objects are achieved by means of a device according to claim 8, preferred variants being defined in the dependent claims.

Yet another object is to provide a method of applying the lock solution of the invention which ensures a significant improvement of the aseptic connection procedure with maintained sterility in the catheter or access system.

An object of the invention is also to provide method that additional simplifies the procedure of rinsing the catheter or access system prior to application of the lock solution.

These objects are also achieved by means of a method in accordance with claim 22, preferred variants being defined in the dependent claims.

Other objects, features, advantages and embodiments of the present invention will become apparent from the following detailed description when taken in conjunction with the drawings and the appended claims.

The device of the invention has a connector which is arranged to prevent the tip of the expulsion arrangement from entering the catheter or other access system lumen, the tip being frangible. With such a device, the tip of the expulsion arrangement may be left behind as a stopper in the connector when the device is removed after injecting the lock solution, thus ensuring maintained sterility.

The connector is preferably a luer lock connector. This type of connector ensures a tight connection and may be fitted on most catheters. However, the connector may of course be of any other equivalent design preventing touch contamination during connection to the catheter.

The frangible tip of the expulsion arrangement may be provided with a peripheral row of indentations. The indentations provide a stress raiser which makes it easy to break off the tip of the plunger.

In order to enhance the engagement of the frangible tip of the expulsion arrangement inside the connector, the frangible tip is preferably provided with a substantially radial projection and an inside of the connector provided with a notch, the projection being arranged to engage the notch.

Another way of enhancing the engagement of the tip of the expulsion arrangement inside the connector is to provide the tip of the expulsion arrangement with a conical shape which tightly fits in an inner conical shape of the connector.

In one embodiment, the housing defines a single compartment which contains the solution to be injected. The one-compartment housing allows a particularly simple construction.

In another embodiment, the housing is divided into a first and second compartment. Thus, two different solutions may be injected using the same applicator device.

The first compartment in a tip end of the housing is preferably filled with flushing solution and the second compartment in a back end of the housing is preferably filled with a lock solution. The applicator device of this embodiment may be used for rinsing a catheter and subsequently applying the lock solution. The flushing solution may be e.g. a saline solution.

The expulsion arrangement may further comprise a divider separating the first and second compartments. This is a way of expelling solution first from the first compartment and then from the second compartment.

According to one embodiment of the invention, the divider is a frangible membrane, and a mandrel at the tip end of the housing is arranged to rupture the membrane. In this manner, the two different solutions may be kept separate during storage and the mandrel ruptures the membrane when the plunger is pressed down to allow solution from the second compartment to pass through the ruptured membrane, into the catheter.

The frangible tip may be arranged on the plunger or on the divider. A suitable placement of the tip may thus be chosen as desired.

As an alternative to a frangible membrane, the device of the invention may comprise a by-pass arranged to shunt the lock solution past the membrane. Thus, lock solution may effectively be injected once the flushing solution has been injected.

In one embodiment of the invention, the divider is a seal including a valve which is openable on pressing down the plunger. This is another way of allowing solution to be expelled from the second compartment into the catheter.

According to the invention, the plunger may be provided with an abutment means for indicating when the first compartment has been emptied. The nurse or physician thus knows when all flushing solution has been inserted, should he/she wish to wait before injecting also the lock solution.

The inventive applicator may advantageously be provided with an air removal system for removing air bubbles.

The air removal system preferably comprises a chamber separated from the atmosphere by an air permeable membrane and arranged to communicate with the catheter when the syringe is connected to the catheter. In this manner, atmospheric pressure may be established in the chamber and blood with air bubbles will flow out into the chamber.

The method of the invention comprises the steps of:

connecting a sterile connector attached to a tip end of a syringe to the catheter or other access system,

injecting the lock solution in the catheter by pressing an expulsion arrangement of the syringe including a plunger, thereby engaging a frangible tip of the expulsion arrangement in the connector,

removing the syringe from the connector, leaving behind the frangible tip of the expulsion arrangement which is broken off when removing the syringe,

closing a lid on the connector.

By using this method lock solution may easily be applied while ensuring maintained sterility in the catheter.

According to a specific variant of the inventive method flushing solution is injected prior to injecting the lock solution. The catheter may thus conveniently be rinsed before application of the lock solution.

In one variant of the method of the invention flushing solution is injected by a first press on the plunger and the lock solution is injected by a second press on the plunger. This is convenient should the nurse or physician wish to wait between rinsing and application of lock solution. An abutment means arranged on the plunger may indicate to the nurse or physician when the flushing solution has been expelled from the syringe.

In another variant the flushing solution and subsequently the lock solution are injected in one continuous press on the plunger. This is a quick way of rinsing the catheter and applying the lock solution.

### SHORT DESCRIPTION OF THE DRAWINGS

Presently preferred embodiments of the present invention will now be described in more detail, reference being made to the enclosed drawings, in which:

FIG. 1 is a diagram showing the proliferation of Staphylococcus epidermidis by measuring the opacity,

FIG. 2 is a diagram showing the proliferation of Staphylococcus epidermidis by measuring the reduction of alamarBlue™,

FIG. 3 is a diagram showing a live/dead bacterial viability assay.

FIG. 4 is a diagram showing the effect of the lock solution according to the invention versus the effect of a solution comprising trypcase soy broth together with different contact surfaces, which could be used in catheters.

FIG. 5 is a perspective view of an applicator device according to a first embodiment of the invention with one compartment with the lock solution of the invention.

FIG. 6 is a cross-sectional view of the applicator device of FIG. 5 shown before the plunger is pressed.

FIG. 7 is a view corresponding to FIG. 6, but shown when the plunger has been pressed all the way down.

FIG. 8 is a perspective view of an applicator device according to a second embodiment of the invention with two compartments.

FIG. 9 is a cross-sectional view of the applicator device of FIG. 8 shown before the plunger is pressed.

FIG. 10 is a view corresponding to FIG. 9, but shown when the plunger has been pressed all the way down.

FIG. 11 is a perspective view of an applicator device according to a third embodiment of the invention with two compartments and a by-pass.

FIG. 12 is a cross-sectional view of the applicator device of FIG. 11 shown before the plunger is pressed.

FIG. 13 is a view corresponding to FIG. 12, but shown when the plunger has been pressed all the way down.

FIG. 14 is a perspective view of an applicator device according to a fourth embodiment of the invention with two compartments and a valve function.

FIG. 15 is a cross-sectional view of the applicator device of FIG. 14 shown before the plunger is pressed.

FIG. 16 is a view corresponding to FIG. 15, but shown when the plunger has been pressed part of the way down and the first compartment has been emptied.

FIG. 17 is a view corresponding to FIG. 15, but shown when the plunger has been pressed all the way down.

FIG. 18 is a perspective view of an applicator device according to a fifth embodiment of the invention with an air removal system.

FIG. 19 is a cross-sectional view of the applicator device of FIG. 18 shown before the plunger is pressed.

FIG. 20 is a view corresponding to FIG. 19, but shown when the plunger has been pressed part of the way down and the first compartment has been emptied.

FIG. 21 is a view corresponding to FIG. 20, but shown when the plunger has been pressed all the way down.

### DETAILED DESCRIPTION

In the medical field glucose degradation products GDP, namely 3-deoxyglucosone (3-DG), acetaldehyde, formaldehyde, glyoxal, methylglyoxal, 5-hydroxymethylfurfurale (5-HMF), are known as antimicrobial agents in different drugs (E. Roux; 1887; Kato, et. al., 1994) because these in turn may react with proteins and lipids to form advanced glycation end-products (AGE) irreversible modifications of these proteins.

The advanced glycation of proteins is a main reaction (Maillard reaction) in food and nutrition biochemistry. It is a non-enzymatic process initiated when proteins are exposed to glucose or other carbohydrates. It generates first reversible Schiff base adducts and subsequently more stable Amadori products. Through a series of oxidation and non-oxidation reactions it yields the irreversible advanced glycation end-products (AGE) linked with amino groups of several proteins. These lead to activation of cell signaling and to DNA damage.

The heat sterilization and storage of conventional peritoneal dialysis solution lead to the formation of these cytotoxic/bactericide GDP (Wieslander, et. al., 1991; 1996).

Besides controlling bacterial growth in the catheter lumen, as proposed by glucose degradation products or elevated glucose concentrations, coagulation of residual proteins at the catheter tip or in the environment of side holes could be avoided by using citrate. Glucose and citrate containing solutions are frequently used e.g. for transfusion medicine as stabilizer agents. By such a solution a mix of well-known compounds for application in humans, e.g. glucose and citrate would be provided. However, this type of formulation has not been proposed so far for lock solutions for medical devices.

The glucose degradation products are normally present in a solution with a carbohydrate concentration of 4% in amounts of about 80 μM for 3,4-DGE, about 500 μM for 3-DG and about 80 μM for 5-HMF. With carbohydrate concentration range of 0.1-50 weight % the GDP ranges are 3-90 μM 3,4-DGE, 15-1800 μM 3-DG and 2-900 μM 5-HMF.

By the invention is proposed an antimicrobial lock solution for medical devices based on carbohydrates and/or glucose degradation products and in one embodiment this lock solution also contains an anticoagulation agent.

The biofunctional properties of the lock solution according to the invention are:

a) antimicrobial, i. e. no growth or proliferation of bacteria. This does not necessarily mean killing of bacteria. It is sufficient to prevent growth if no or low counts of bacteria are instilled in the catheter lumen, especially in a situation where the surface of the catheter does not allow adherence of bacteria or biofilm formation.

b) anticoagulatory, i. e. no fibrin net works or platelet aggregates should be formed at the catheter surface. There are two problems associated with clotting: (i) propagation of occlusion with subsequent reduction in blood flow and increase in pressure and (ii) fibrin net or platelet aggregates are known to serve as growth substrate for bacteria in a secondary step.

The lock solution of the invention is intended to be applied after rinsing of the catheter after treatment and keeping an anticoagulatory as well as antimicrobial environment within the catheter. A single measure in access care, e.g. an antimicrobial surface only is not sufficient to realize a clinically significant effect.

When using a lock solution the anticoagulant is drained out from the catheter and about 20 ml saline is entered into the catheter void of 2.5 ml and is pushed back and forth in the catheter. The lines are connected and the dialysis performed. After dialysis the artery line is disconnected, and rinsed with 20 ml saline once, the venous line is disconnected and also rinsed with 20 ml saline. Thereafter both parts are filled with heparin as an anticoagulant. By using this technique only a minimal volume of the lock solution is released into the blood stream.

Examples of substances having anticoagulatory properties which may be used according to the invention are e. g. inhibitors of the coagulation cascade such as heparin of standard and low molecular weight, fractionated heparin, synthetic inhibitors in the coagulation cascade, Futhan as a broad protease inhibitor, complexing and chelating substances such as citrate, EDTA, EGTA, substances and mixtures used for preservation of blood products (platelets or plasma), CDPA (citrate, sodium phosphate, dextrose, adenine), synthetic or natural thrombin inhibitor substances.

Carbohydrates and glucose degradation products containing citrate solution is a preferred solution. Citrate in concentrations above 10% may not be safe as even small amounts of citrate entering the right atrium of the heart can cause a local reduction of in calcium ions in the heart muscle. Food Drug Administration (FDA) has issued a warning against the use of citrate in concentrations above 4 weight %. Preferably, according to the invention, the concentration of citrate is less than 4 weight %.

In addition the lock solution may be combined with other additives which have not been considered for lock solutions so far, e. g. fucosidan, and others.

Substances such as vitamins and nutritional additives could be preformulated in a catheter fluid applicator in elevated or tailored concentrations and having anticoagulatory properties, e. g. riboflavin, vitamin E, alpha-tocopherol, folic acid and amino acids. Furthermore, antiinflammatory compounds and drugs could also be used, e.g. cortison, mycophenolic acid (MPA) and derivates thereof, sirolimus, tacrolimus and cyclosporin, diclofenac, etc.

Also inhibitory peptides such as defensins, (dermacidine), and others may be used in the lock solution. Radicals, such as reactive oxygene species, NO-releasing systems or nitric oxide (NO), and peroxynitrite may also be used. A buffer composition is preferably made which may comprise lactate, bicarbonate, pyruvate, ethyl pyruvate and citric acid in combination and mixtures including adjustment of pH by acetic acid, hydrochloric acid or sulphuric acid.

Furthermore, viscosity enhancing additives may be added, such as lipids or lipidic substances (also to get water insoluble vitamins or complexes into solution), nutrients in high concentration density gradient e.g. aminoacid containing fluids, polyglucose, Icodextrin, pectine, hydroxyethyl starch (HES), alginate, hyaluronic acid, etc.

However, to solve the problems of catheter care by offering a fluid only would not help to a full extent in the practical handling of catheters. For a safer application an applicator may be provided which consists of a syringe prefilled with the lock solution of the invention. The technique consists of instilling the lock solution into the catheter lumen after each application and to withdraw it before subsequent application.

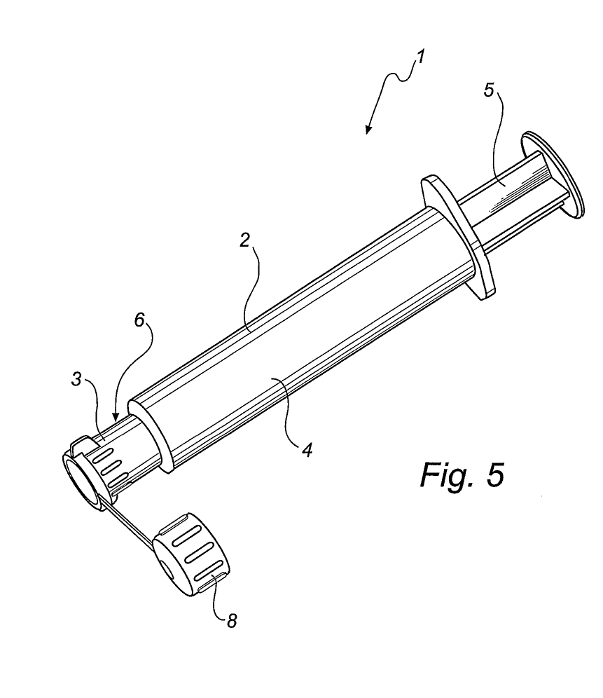

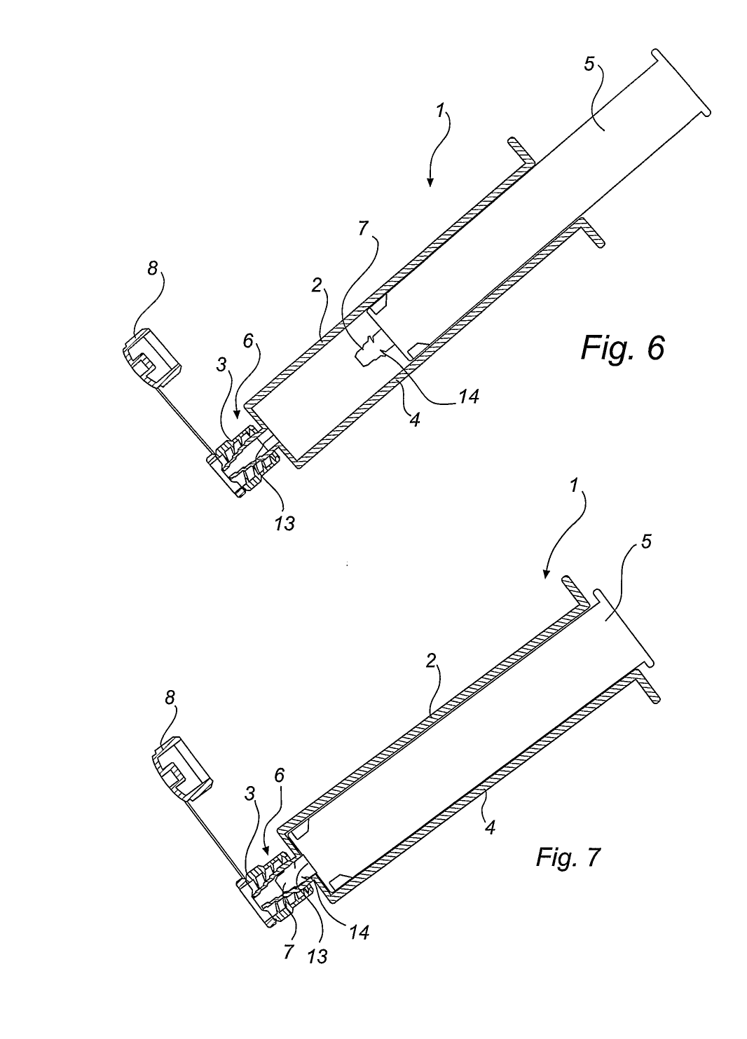

The applicator device 1 of FIGS. 5, 6 and 7 basically consists of a hollow body similar to a syringe 2 provided with a connector in the form of a connector 3 for connection with a catheter or other access system (not shown). The syringe 2 has an elongate housing 4 in which a plunger 5 is coaxially arranged. The plunger 5 constitutes an expulsion arrangement for expelling a lock solution according to the invention from the syringe. On a tip end 6 of the housing 4 the connector 3 is connected. The plunger 5 has a tip 7 which is frangible.

When the catheter has been disconnected from e.g. a dialysis machine or other bloodline system, it is important to make sure that no blood clots are formed in the catheter and that microorganisms are prevented from entering the catheter. Therefore, the catheter is rinsed by means of a separate syringe filled with flushing solution, e.g. saline solution. Once the catheter has been rinsed, a lock solution according to the invention may be applied in the lumen of the catheter by means of the applicator device 1. The connector 3 is connected to the catheter and the tip end 6 of the housing 4 is fixed inside the connector 3. When the plunger 5 is pressed down, the lock solution enters the catheter. As the plunger 5 is pressed all the way down the tip 7 is stuck inside the connector 3. The inner shape of the connector 3 and the outer shape of the tip 7 ensure that the tip 7 does not enter the catheter. This may be achieved e.g. by means of projections on the outside of the tip 7 and corresponding notches on the inside of the connector 3 or preferably by the tip being shaped as a cone fitting in an inner cone shape of the connector 3. The tip 7 is provided with a stress raiser in the form of a peripheral row of punctures or indentations 14. Once the tip 7 is stuck inside the connector 3, the syringe 2 may be withdrawn and the broken-off tip 7 left in the connector 3, closing the opening of the connector 3. The tip end 6 of the housing 4 is also broken off and left together with the connector 3. When the syringe 2 has been removed, a lid 8 is placed over the connector 3, which remains connected to the catheter. In this manner, a lock solution is applied in the catheter while maintaining the sterility of the opening of the catheter.

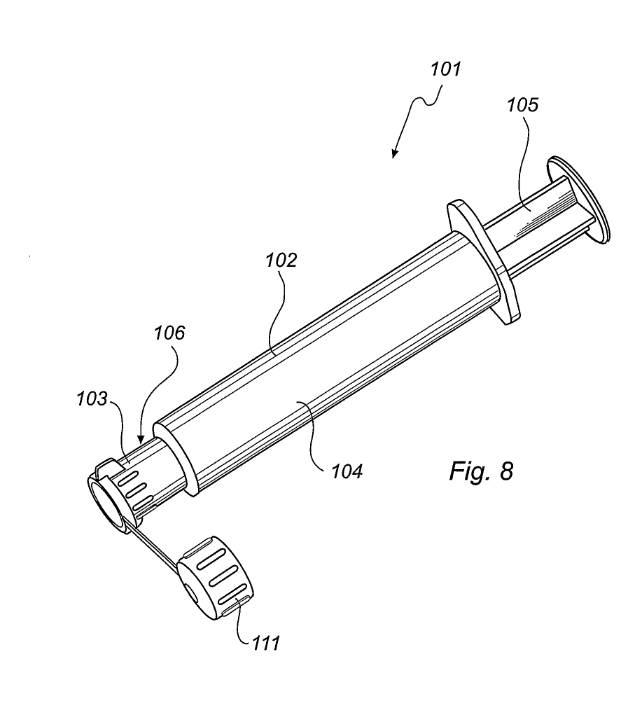

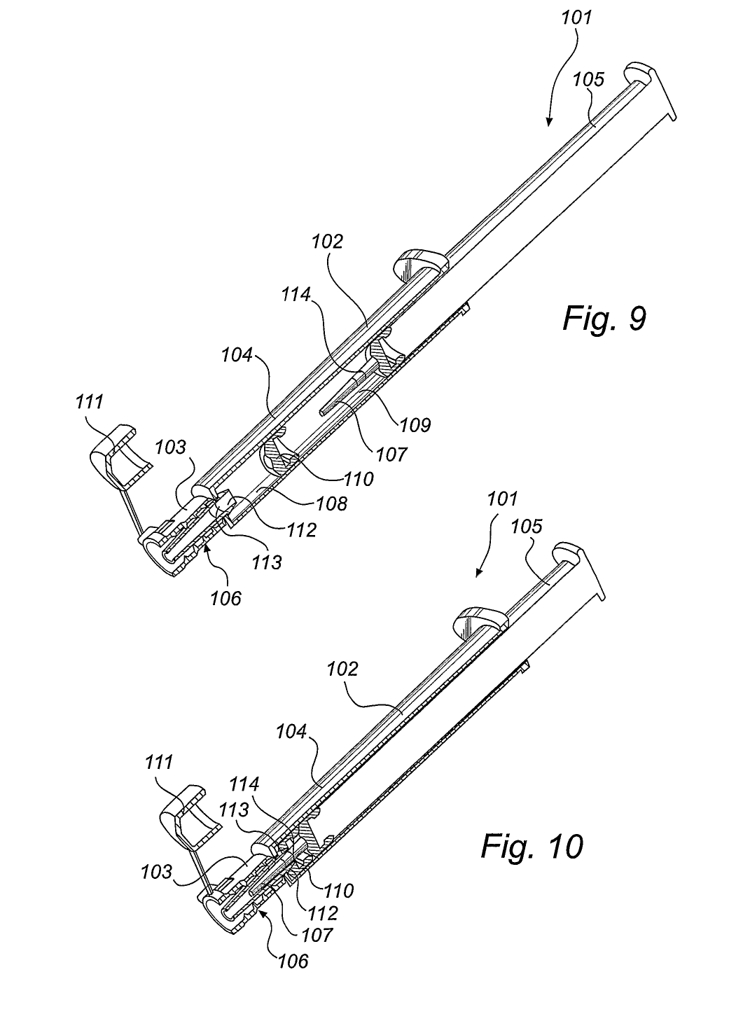

In the embodiment of FIGS. 8-10, the applicator device 101 is similar to the applicator device 1 of FIG. 5, except that the applicator device 101 has a housing 104 which is divided into two compartments 108, 109 delimited by a divider in the form of a frangible membrane 110. With the plunger 105 the membrane 110 forms an expulsion arrangement for expelling solution form the syringe. The tip end compartment 108 is filled with flushing solution, e.g. saline solution, and the back end compartment 109 is filled with a lock solution according to the invention. A frangible tip 107 is attached to the plunger 105. The tip end 106 of the housing 104 is provided with a mandrel 112 arranged in a semicircle. The mandrel 112 has a sharp forward cutting edge for rupturing the membrane 110.

As with the applicator device 1 of FIG. 5, the applicator device 101 is connected to a catheter or other access system via the connector 103. As the plunger 105 is pressed, first the saline solution of the first compartment 108 is injected into the catheter. When all saline solution has been injected, the membrane 110 has reached the tip end 106 of the housing 104. The membrane 110 is then ruptured by the mandrel 112 and continued pressing of the plunger 105 injects the lock solution of the second compartment 109. As the plunger 105 is pressed all the way down, the tip 107 engages the inside of the connector 103 in the same way as in the first embodiment, as can be seen in FIG. 10. Therefore, as the syringe 102 is removed, the tip 107 and the tip end 106 of the housing 104 are left behind, closing the opening of the catheter. As with the first embodiment, this embodiment ensures that sterility is maintained in the catheter. Furthermore, the applicator device 101 of this second embodiment simplifies the rinsing and locking of the catheter since it obviates the need for a separate syringe for the flushing solution. When the syringe 102 has been removed, a lid 111 is placed over the connector 103.

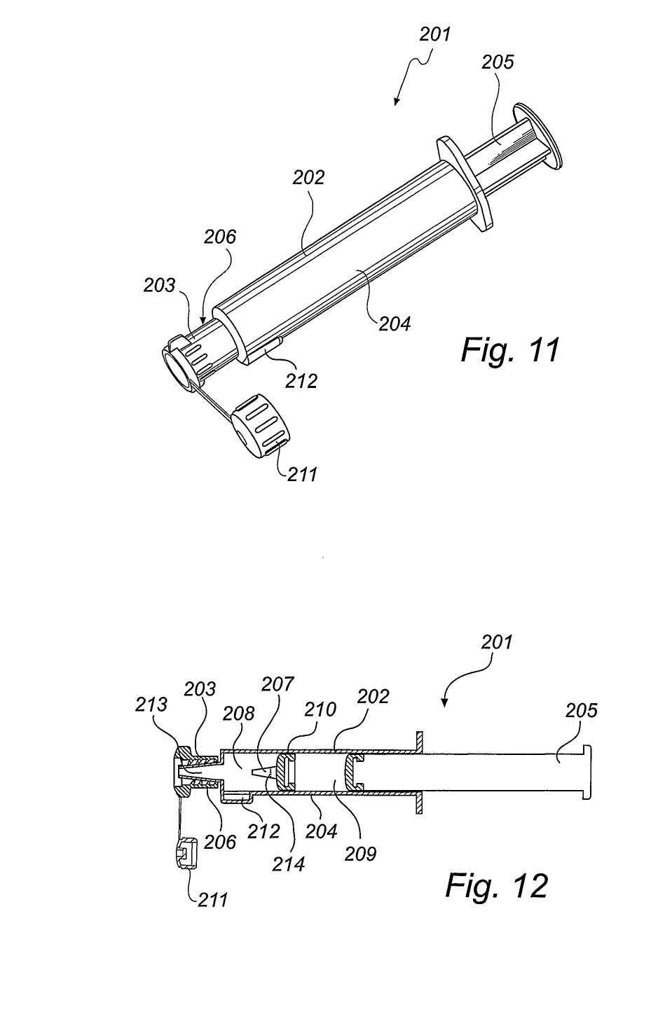

In the embodiment of FIGS. 11-13 the applicator device 201 is similar to the applicator device 101 of FIG. 8, but is provided with a by-pass 212 near the tip end 206 of the housing 204, and not provided with a mandrel. Just as in the device 101, the plunger 205 and a divider, in this case in the form of a non-frangible membrane 210, form an expulsion arrangement for expelling solution from the syringe 202. In contrast to the device 101, the frangible tip 207 is arranged on the membrane 210 and not on the plunger 205.

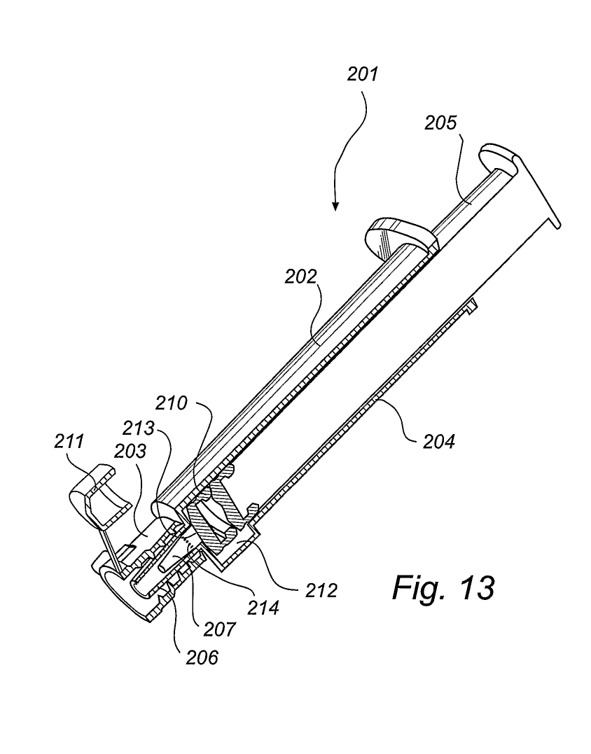

As with the applicator devices of FIGS. 5 and 8, the applicator device 201 is connected to a catheter via the connector 203. As the plunger 205 is pressed, first the flushing solution (e.g. saline solution) of the first compartment 208 is injected into the catheter. When all saline solution has been injected, the membrane 210 has reached the tip end 206 of the housing 204. Continued pressing of the plunger 205 injects the lock solution of the second compartment 209. Since the membrane 210 blocks the outlet passage 213 at the tip end 206 of the housing 204, the lock solution by-passes the membrane 210 via the by-pass 212. As the plunger 205 is pressed all the way down, the tip 207 which is attached to the membrane 210 is pushed all the way down and engages the inside of the connector 203 in the same way as in the first and second embodiments, as can be seen in FIG. 13. Therefore, as the syringe 202 is removed, the tip 207 is left behind. The tip end 206 of the housing 204 is also broken off and left behind. Just as with the first and second embodiments, this embodiment ensures that sterility is maintained in the catheter. Furthermore, the applicator device 202 of this third embodiment simplifies the rinsing and locking of the catheter since it obviates the need for a separate syringe for the flushing solution. When the syringe 202 has been removed, a lid 211 is placed over the connector 203.

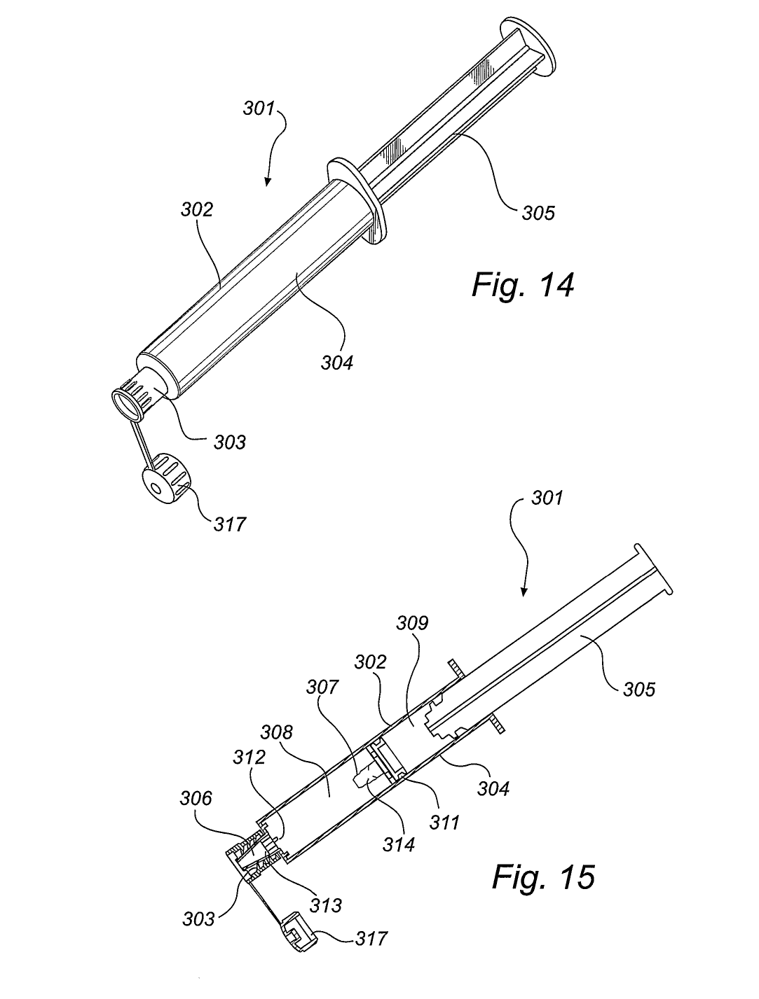

In the embodiment of FIGS. 14-17, the applicator device 301 is similar to the applicator devices of FIGS. 8 and 11, except that the two compartments 308, 309 are delimited by a seal 311. With the plunger 305, the seal 311 forms an expulsion arrangement for expelling solution from the syringe 302. As in the second and third embodiments, the tip end compartment 308 is filled with flushing solution (e.g. saline solution) and the back end compartment 309 is filled with a lock solution according to the invention. The seal 311 that separates the two compartments 308, 309 is provided with a frangible tip 307 corresponding to the tip 7 in the first embodiment.

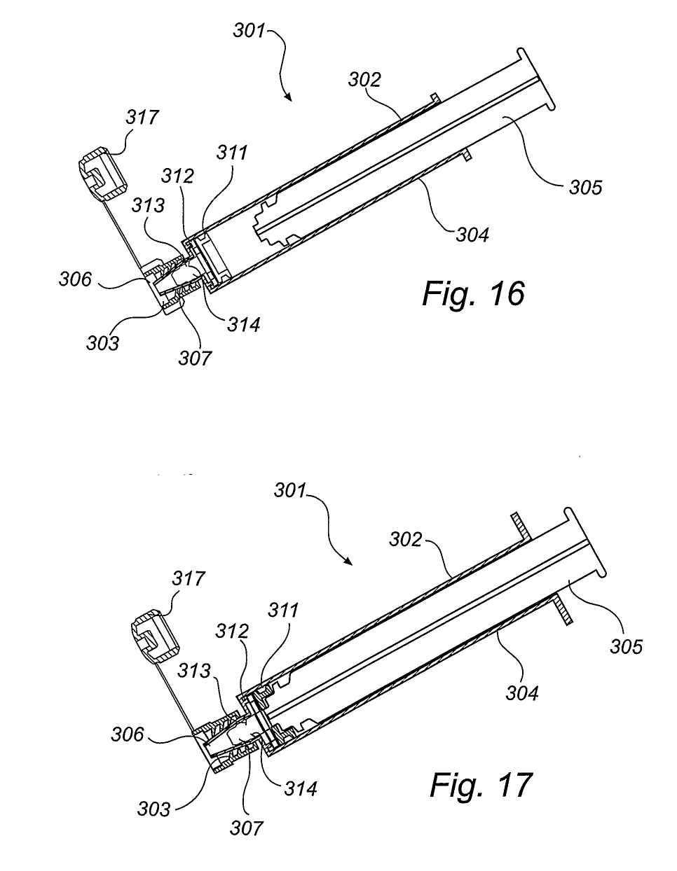

As with the applicator devices of the other embodiments, the applicator device 301 is connected to a catheter or other access system via the connector 303. As the plunger 305 is pressed, first the flushing solution of the first compartment 308 is injected into the catheter. When all flushing solution has been injected, the seal 311 has reached the tip end 306 of the housing 304, as can be seen in FIG. 16. Through continued pressing of the plunger 305 a valve in the seal is opened and the lock solution of the second compartment 309 can be injected. The valve in the seal 311 is constituted by slits in the seal 311, which are normally closed, but which open when the seal impacts spacers 312 at the tip end 306 of the housing 304. As the plunger 305 is pressed all the way down, the tip 307 is also pressed all the way down and engages the inside of the connector 303 in the same way as in the other embodiments, as can be seen in FIG. 17. Therefore, as the syringe 302 is removed, the tip 307 is left behind, closing the opening of the catheter. The tip end 306 of the housing 304 is also broken off and left in the connector 303. As with the three other embodiments, this embodiment ensures that sterility is maintained in the catheter. Furthermore, the applicator device 302 of this fourth embodiment simplifies the rinsing and locking of the catheter since it obviates the need for a separate syringe for the flushing solution. When the syringe 302 has been removed, a lid 317 is placed over the opening of the connector 303.

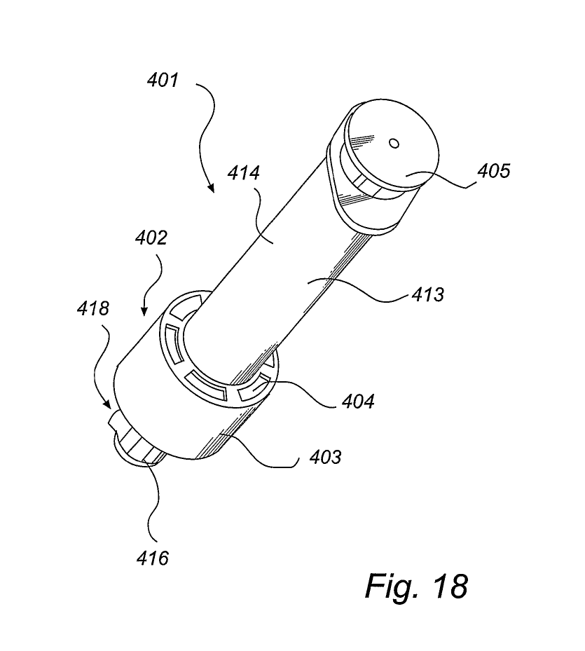

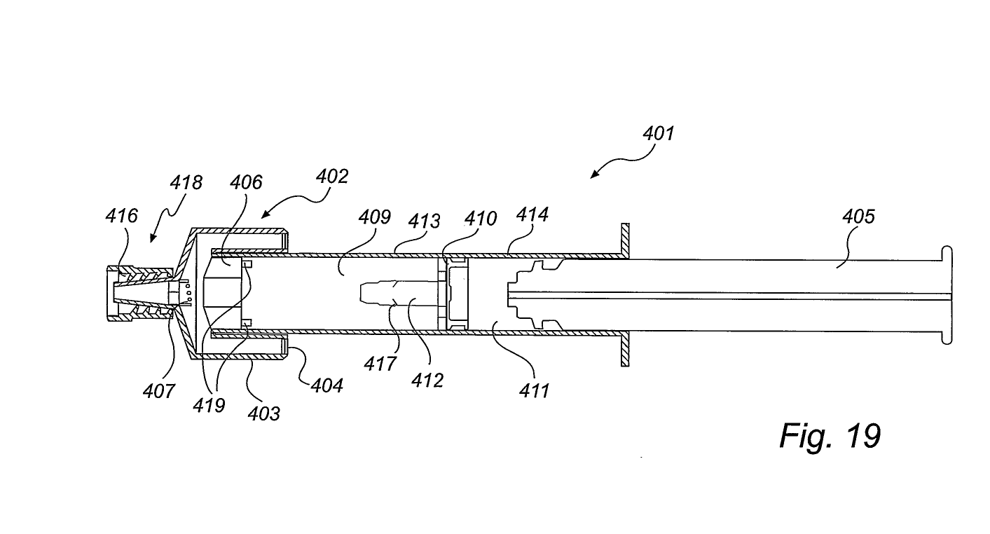

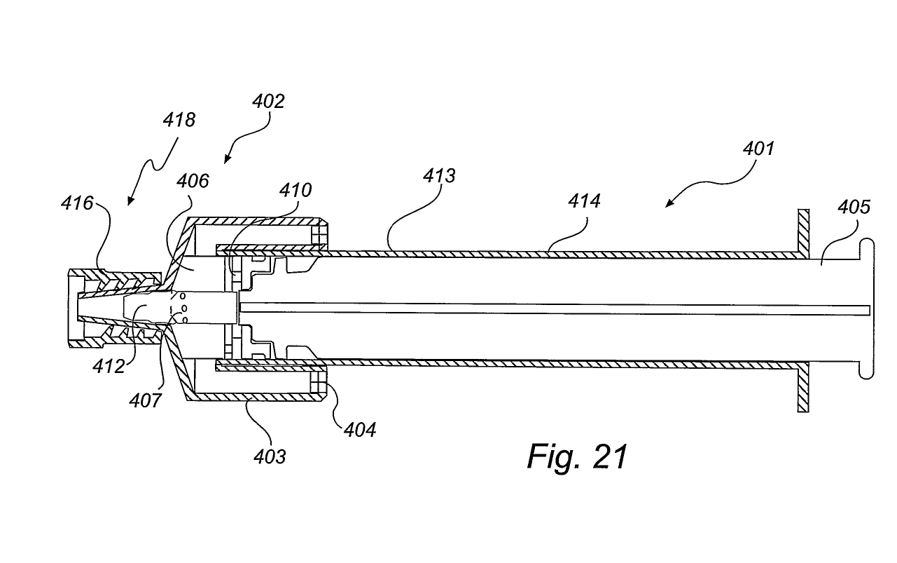

In the embodiment of FIGS. 18-21, the applicator device 401 is similar to the applicator device of FIGS. 14-16, except that this fifth embodiment includes an air removal system 402, which consists of a separate chamber 403 of the applicator 401 with an air permeable membrane 404, such that the pressure in the chamber 403 is similar to the atmospheric pressure p₂ outside the applicator 401. When the catheter is connected with the applicator 401, the air removal system 402 communicates with the catheter. The blood pressure p₁ in the catheter is higher than the pressure p₂ in the chamber 403. The blood with air bubbles will therefore flow into the chamber 403. When the chamber is filled with blood, the plunger 405 is pressed down, so that the stopper 406 moves downwards and becomes penetrated by the mandrel 407. When the stopper 406 is pressed all the way down, the blood with air is enclosed in the chamber 403. By pressing the plunger 405 further down, the rinsing solution in the forward compartment 409 is pressed into the catheter. With the plunger 405, the seal 410 forms an expulsion arrangement for expelling solution from the applicator 401. The back end compartment 411 is filled with a lock solution. The valve 410 that separates the tip end compartment 409 with the rinsing solution from the back end compartment 411 is provided with a frangible tip 412.

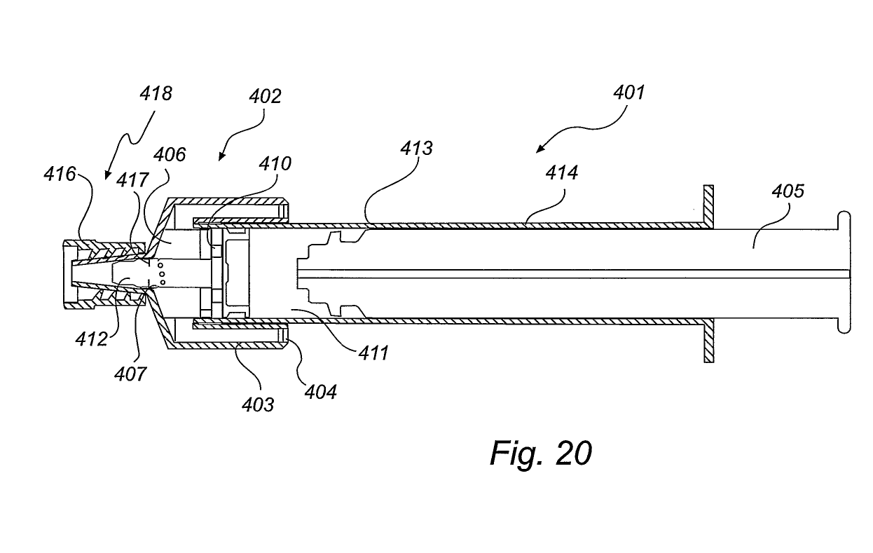

When all rinsing solution has been injected into the catheter, the valve 410 has reached the tip end of the housing, as can be seen in FIG. 20. By continued pressing of the plunger 405, a valve 410 in the seal is opened and the lock solution of the back end compartment 411 can be injected into the catheter. The valve 410 in the seal is constituted by slits in the seal, which are normally closed, but which open when the seal impacts spacers 419 at the tip end of the housing. As the plunger 405 is pressed all the way down, the tip 412 is also pressed all the way down and engages the inside of the tip end of the housing and the luer lock in the same way as in the other embodiments described above. Therefore, as the syringe 413 is removed, the tip 412 is left behind, closing the opening of the catheter. The tip end of the housing is also broken off and left in the luer lock.

The skilled person realizes that a number of modifications of the embodiments described herein are possible without departing from the scope of the invention, which is defined in the appended claims.

For instance, the two-compartment applicator device 101; 201; 301 may be provided with a small abutment on the plunger 105; 205; 305, so that the nurse or physician is given an indication when the tip end compartment 108; 208; 308 has been emptied. The plunger 105; 205; 305 may then be pressed further, past the abutment, for emptying the back end compartment. Otherwise, the injection of the flushing solution and the lock solution may be done in one continuous push.

The two-compartment applicator device 101; 201; 301 or an applicator with more than two compartments, may also be suitable in cases where the components of the lock solution need to be stored separately during sterilization and storage. In such cases, distilled water or a simple buffer solution is contained in one compartment and other components in dry form or in high concentrations are contained in the other compartment(s).

### EXAMPLES

In table 1 below different solutions in accordance with the invention are shown. Solution A is a ACD solution in transfusion medicine, solution B is a CPDA solution in blood products, solution C is a conventional solution for peritoneal dialysis and solutions D are conventional solutions acc. Col III with lower or high glucose concentration.

TABLE 1

| A | B | C | D |
|---|---|---|---|
| 3.19% glucose | 3.27 g/L acidic | 4.0% glucose | 0.1 to 50% |
| 1.04% citric | citric | 5.4 g/L sodium | |
| acid | monohydrate | chloride | |
| monohydrate | 26.3 g/L sodium | 0.199 g/L | |
| 2.87% sodium | citrate | calcium | |
| citrate | 2.51 g/L sodium | chloride | |
| dehydrate | dihydrogen | 0.051 g/L | |
| | phophate | magnesium | |
| | dihydrate | chloride | |
| | 31.9 g/L | 4.5 g/L sodium | |
| | dextrose | lactate | |
| | monohydrate | anhydrous | |
| | 275 g/L adenine | hydrochloric | |
| | | acid | |
| | | pH 5.5 | |

In table 2 different solutions of preferred compositions of carbohydrates, carbonyl compounds and citrate are shown:

The preferred solution composition allows complete infusion of the lock media into the body/blood stream with out any harmful effects as they are diluted in the circulating blood, quickly resorbed, metabolized or even of therapeutic or nutritional support.

TABLE 2

| | | Solution 3 | Solution 4 | Solution 5 |
|---|---|---|---|---|
| | | Carbo- | Carbo- | Carbo- |
| Solution 1 | | hydrates/ | hydrates/ | hydrates/ |
| Carbo- | Solution 2 | GDP/ | GDP/ | GDP/ |
| hydrates/ | Carbohydrates/ | citrate/ | citrate/ | citrate/ |
| GDP/citrate | GDP/citrate | additive 1 | additive 2 | additive 3 |
| 4.0% | 4% fructose | 4.0% | 4.0% | 4.0% |
| glucose | or other | glucose | glucose | glucose |
| 5.4 g/L | carbohydrates | 5.4 g/L | 5.4 g/L | 5.4 g/L |
| sodium | 5.4 g/L | sodium | sodium | sodium |
| chloride | sodium | chloride | chloride | chloride |
| 0.199 g/L | chloride | 0.199 g/L | 0.199 g/L | 0.199 g/L |
| calcium | 0.199 g/L | calcium | calcium | calcium |
| chloride | calcium | chloride | chloride | chloride |
| 0.051 g/L | chloride | 0.051 g/L | 0.051 g/L | 0.051 g/L |
| magnesium | 0.051 g/L | magnesium | magnesium | magnesium |
| chloride | magnesium | chloride | chloride | chloride |
| 4.5 g/L | chloride | 4.5 g/L | 4.5 g/L | other buffer |
| sodium | 4.5 g/L | sodium | sodium | system |
| lactate | sodium | lactate | lactate | pH 5.5 |
| anhydrous | lactate | anhydrous | anhydrous | 3.8% |
| hydrochloric | anhydrous | hydrochloric | hydrochloric | citrate |
| acid | hydrochloric | acid | acid | |
| pH 5.5 | acid | pH 5.5 | pH 5.5 | |
| 3.8% | pH 5.5 | 3.8% | 3.8% | |
| citrate | 3.8% | citrate | citrate | |
| | citrate | riboflavin | viscosity | |
| | | as vitamin | enhancing | |
| | | type of | compounds | |
| | | additive | | |

Solution 6/7/8=3/4/5 but also with fructose or other carbohydrates.

Solution 9/10/11/12=1/2/3/4 but with buffer system like solution 5.

Solution 13-24=the same solutions as 1-12 but with mixed carbohydrates.

Solutions 25-68=the same solutions as 1-24 but with additional of antimicrobial additives.

The carbohydrate concentration may be higher: 0.1-50%.

Glucose can be partially replaced by fructose or other sugars which can be metabolized by the human body.

Riboflavin (<500 μmol) and other vitamins may further be used as additives. Viscosity enhancing additives:

Besides the properties of glucose and GDPs to limit bacterial growth elevated concentrations are able to contribute to enhanced viscosity. This is advantageous in the application of a lock solution to prevent continuous bleeding out of the lock medium. Other additives in the context could be polyglucose molecule, lipids and the like.

Different investigations were made with the solutions regarding the proliferation of bacteria, the viability of the bacteria and the toxicity. As bacteria strain, Staphylococcus epidermidis (ATCC 12228) was chosen because it is known that these are the main microbes which are responsible for catheter related bloodstream infections.

Proliferation of Bacteria

Different methods were developed for testing bacterial proliferation:

Nephelometry

The proliferation of bacteria were tested with nephelometry. Here the bacteria density is compared to a series of standards of different opacities called McFarland. A densitometer is applied to measure the bacterial density produced in an ampoule of liquid medium. It gives values in McFarland units, proportional to the average values of bacterial concentration obtained with gram-negative rods isolated from clinical specimens.

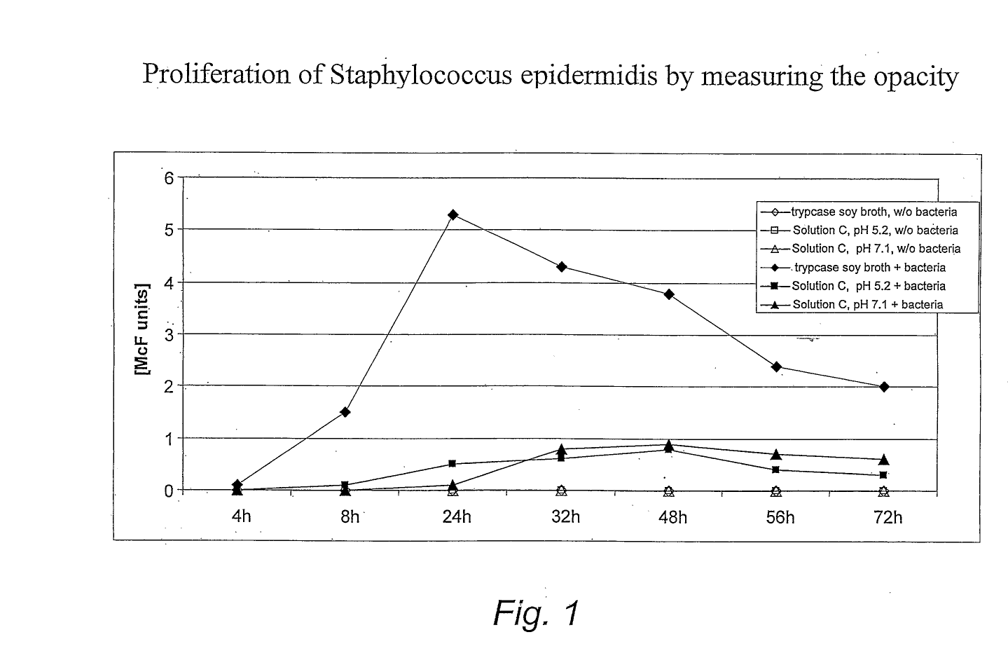

In FIG. 1 the results show reduced bacterial proliferation in a solution according to solution C in table 1, independent on pH in comparison to normal trypcase soy broth. These experiments were repeated several timed with the same results. The decrease of proliferation of Staphylococcus epidermidis over time in trypcase soy broth results from deficiency of nutrients. From FIG. 1 it is clearly evident that the lock solution according to the invention is an antiproliferation agent for bacteria.

alamarBlue™ Assay

The proliferation of bacteria was also tested with the alamarBlue™ assay. This assay is designed to measure quantitatively the proliferation of various human and animal cell lines, bacteria and fungi.

The alamarBlue™ assay incorporates a fluorometric/colorimetric growth indicator based on detection of metabolic activity. Specifically, the system incorporates an oxidation-reduction (REDOX) indicator that both fluoresces and changes color in response to chemical reduction of growth medium resulting from cell growth.

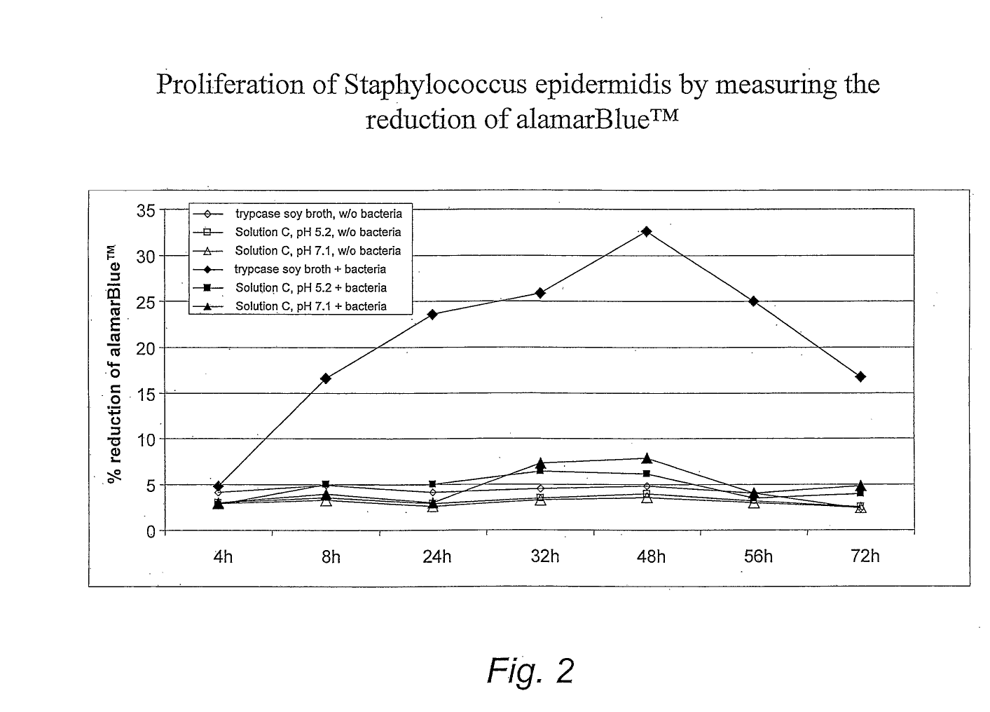

As the cells or bacteria being tested grow, innate metabolic activity results in a chemical reduction of alamarBlue™. Continued growth maintains a reduced environment while inhibition of growth maintains an oxidized environment. Reduction related to growth causes the Redox indicator to change from oxidized (non-fluorescent, blue) form to reduced (fluorescent, red) form. In FIG. 2 the results from measuring the proliferation with alamarBlue™ show the same amount in inhibition of growth as by measuring the opacity. Also here a solution according to solution C in table 1 were used for the tests and the tests were made independent on pH in comparison to normal trypcase soy broth. These experiments were repeated several timed with the same results.

Viability of Bacteria

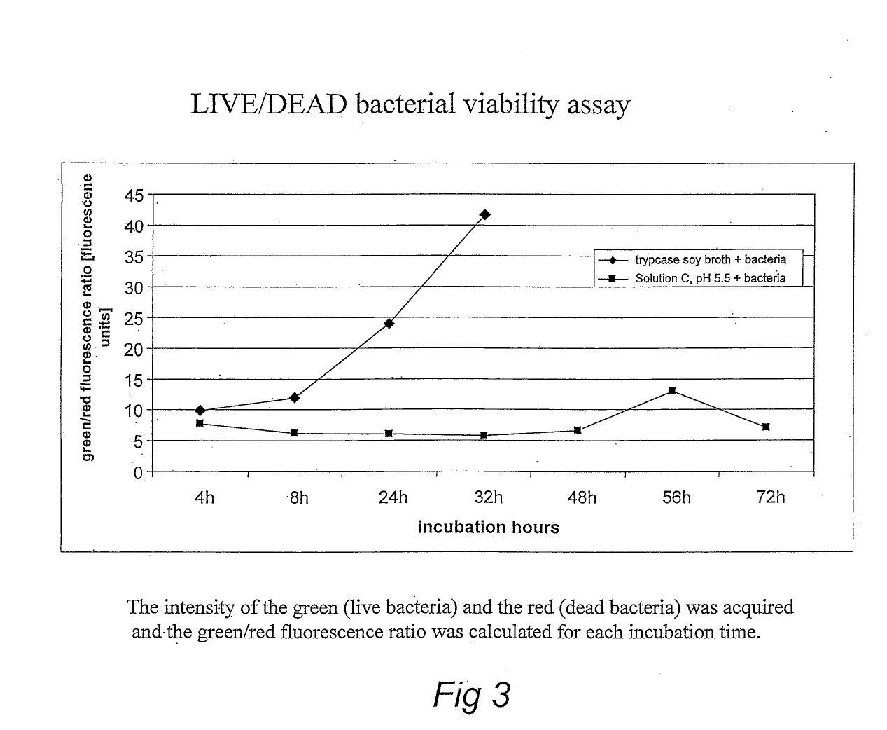

The viability of the bacteria was tested by the use of LIVE/DEAD BacLight™ Bacterial Viability Kit. The kit utilized mixtures of SYTO 9 green-fluorescent nucleic acid stain and red-fluorescent nucleic acid stain, propidium iodide. These stains differ both in their characteristics and in their ability to penetrate healthy bacterial cells. When used alone the SYTO 9 Stain generally labels all bacteria in a population—those with intact membranes and those with damaged membranes. In contrast propidium iodide penetrates only bacteria with damaged membranes, causing reduction in the SYTO 9. With a mixture of SYTO 9 and propidium iodide stains, bacteria with intact cell membranes stain fluorescent green, whereas bacteria with damaged membranes stain fluorescent red. The maximum excitation/emission for these dyes are about 480 nm/500 nm for SYTO 9 and 490 nm/635 nm for propodium iodide. In FIG. 3 the results from the LIVE/DEAD BacLight™ Bacterial Viability test show an antiproliferative effect of the solution C in table 1 above which is also shown in the other investigations.

Proliferation of Bacteria on Different Surfaces

To consider if the combination of both an antimicrobial surface and the lock solution according to the invention lead to reduced bacterial infection in a catheter or other access system and this results in a better outcome, synonymous with e.g. longer dwelling time of the catheter or other access system some tests were run using different catheter surfaces in combination with different solutions.

To evaluate the antibacterial activity of the surface, films from the following coating solution were investigated.

The following formulations were investigated:

| | | Coating Recipe [weight %] | | | |
|---|---|---|---|---|---|
| No. | Name | PUR | MIBK | SMA | Bismuth |
| 1 | PUR | 40 | 60 | 0 | 0 |
| 2 | PUR-SMA | 35 | 60 | 5 | 0 |
| 3 | PUR-0.03% Bi | 40 | 60 | 0 | 0.03 |
| 4 | PUR-0.05% Bi | 40 | 60 | 0 | 0.05 |
| 5 | PUR-SMA-0.03% | 35 | 60 | 5 | 0.03 |
| | Bi | | | | |
| 6 | PUR-SMA-0.05% | 35 | 60 | 5 | 0.05 |
| | Bi | | | | |

SMA: Tegomer H—Si 6440 (Goldschmidt)
MIBK: Methylisobutylketon (Fluka (58600))
PUR: Desmodur E23, (Bayer)
Bismuth: Triphenylbismuthdichlorid (Aldrich)

To evaluate the antibacterial activity of the solution, normal trypcase soy broth as a positive control was compared with the solution C in table 1.

The concentration of bacteria was determined with nephelometry as disclosed earlier.

The test was begun by seeding a concentration McF=0.1 of Staphylococcus epidermidis (ATCC 12228) in a trypcase soy broth or in the lock solutions into 24-well-plate (1 ml/well; minimum in triple) glued with the different films and was incubated up to 48 hrs at 37° C. in an incubation chamber without CO₂.

After the incubation time the supernatant was removed and the films were tested for bacterial adhesion and proliferation using the alamarBlue™ assay, disclosed above following the protocol from the supplier.

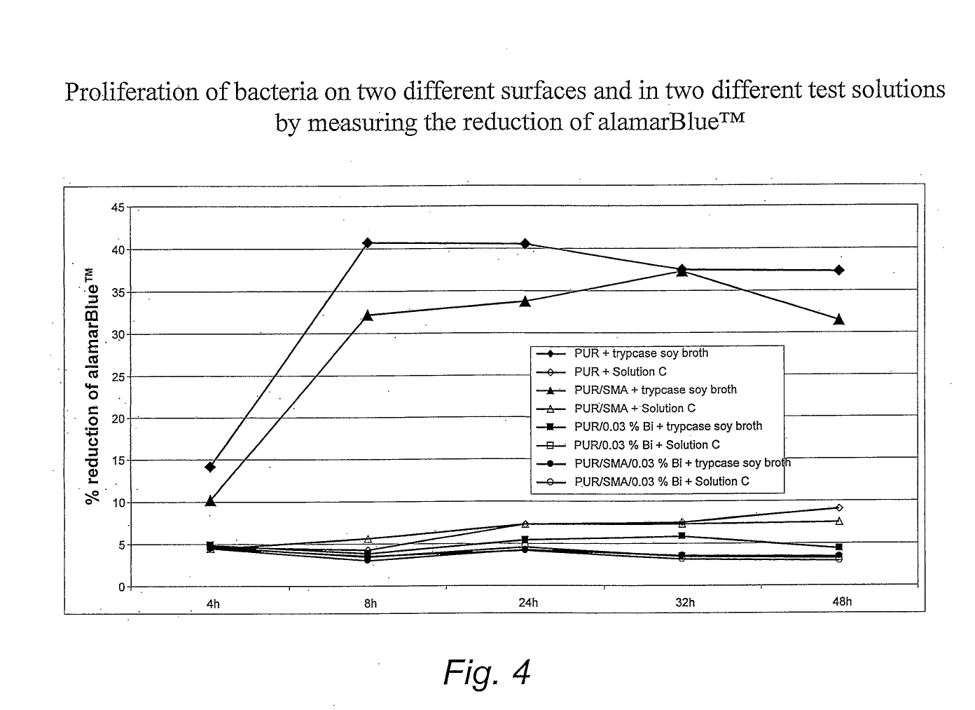

FIG. 4 shows the proliferation of bacteria on two different coated but non-antimicrobial surface (=PUR and PUR/SMA) and on two different coated antimicrobial surface (=PUR/0.03% Bi and PUR/SMA/0.03% Bi) once in the normal trypcase soy broth and once in the lock solution C of table 1, which also was used in the other tests.

These results show that on the non-antimicrobial surfaces the bacterial adherence and proliferation in trypcase soy broth results in an exponential growth rate but on anti-microbial surfaces the bacterial adherence and proliferation in trypcase soy broth is inhibited.

Growing of the bacteria in the solution C instead of trypcase soy broth results also in growth inhibition.

The diagram shows also that the antimicrobial surface PUR/SMA/0.03% Bi have a high potential in the first 48 hrs regarding adherence and thus proliferation of bacteria. But additional incubation of the bacteria in an antimicrobial solutions could cause in a growth inhibition over time because each surface have a limited “non-adherence” potential. But it is also shown in these diagram that on an antimicrobial surface (PUR/0.03% Bi) which have not these exceeding antimicrobial effect like PUR/SMA/0.03% Bi adherence and thus proliferation can additional be reduced by using an antimicrobial solution as incubation media.

Based on our results we can conclude that both together the antimicrobial surface and the antimicrobial solution results in a strong decrease of bacterial adherence and proliferation. With such a combination catheter related blood stream infection could be minimized and should result in a better clinical outcome.

1. A lock solution for medical devices comprising carbohydrates, glucose degradation products, or a combination of carbohydrates and glucose degradation products.

2. A lock solution according to claim 1, wherein the carbohydrates are chosen from the group of glucose and fructose.

3. A lock solution according to claim 1 or 2, wherein the glucose degradation products are chosen from the group of 3-deoxyglucosone, acetaldehyde, formaldehyde, glyoxal, methylglyoxal, 5-hydroxymethyl-2-furaldehyde, 2-fluraldehyde, and 3,4-dideoxyglucosone-3-ene.

4. A lock solution according to claim 1, wherein the carbohydrates, the glucose degradation products, or the combination of carbohydrates and glucose degradation products are antimicrobial agents, said carbohydrates, glucose degradation products, or combination of carbohydrates and glucose degradation products being the sole antimicrobial agents in the lock solution.

5. A lock solution according to claim 1, further comprising an anticoagulation agent.

6. A lock solution according to claim 5, wherein the anticoagulation agent is citrate.

7. A lock solution according to claim 6, wherein the concentration of citrate in the lock solution is less than 4 weight %.

8. A lock solution according to claim 1, wherein the concentration of the carbohydrates is 0.1-50 weight %.

9. A lock solution according to claim 1, wherein the glucose degradation products comprise 3,4-3,4-dideoxyglucosone-3-ene having a concentration of 3-90 μM, 3-deoxyglucosone having a concentration of 15-1800 μM, and 5-hydroxymethyl-2-furaldehyde having a concentration of 2-900 μM.

10. A lock solution for medical devices comprising carbohydrates and glucose degradation products, said carbohydrates being chosen from the group of glucose and fructose and having a concentration of 0.1-50 weight %.

11. A lock solution according to claim 10, wherein the glucose degradation products comprise 3,4-3,4-dideoxyglucosone-3-ene having a concentration of 3-90 μM, 3-deoxyglucosone having a concentration of 15-1800 μM, and 5-hydroxymethyl-2-furaldehyde having a concentration of 2-900 μM.
